(19) United States
(12) Patent Application Publication
(10) Pub. No.: US 2004/0254202 A1
(43) Pub. Date: Dec. 16, 2004

(54) Method of promoting sleep using topical administration of vasoactive agents

(75) Inventor: James Patrick Ebel, Lebanon, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/461,701

(22) Filed: Jun. 13, 2003

(51) Int. Cl. A61K 31/522; A61K 31/455; A61K 31/4415; A61K 31/355

(52) U.S. Cl. 514/263.32; 514/350; 514/355; 514/458

(57) ABSTRACT

The present invention is directed to a method of promoting sleep by topically applying onto the skin a composition that comprises a non-hypnotic, vasoactive agent. The non-hypnotic, vasoactive agent is highly effective in providing for the improved quality of sleep through the use of topical application.

### FIELD OF THE INVENTION

The present invention is directed to a method of promoting sleep using vasoactive agents that are topically applied. In particular, the present invention is directed to a method for providing sleep benefits wherein the method comprises topically administering compositions that comprise non-hypnotic, vasoactive agents.

### BACKGROUND OF THE INVENTION

Sleep disorders can be described to involve symptoms of the inability to fall asleep, interrupted sleep, insomnia, lack of restfulness, on and off sleep, and the tired, sluggish, or exhaustive feeling due to poor sleep and/or the lack of sleep. In order to alleviate symptoms related to sleep disorders, there exist products such as sleeping pills and liquid formulations comprising a sleep agent. Sleeping pills and liquid sleep formulations have been shown to provide some benefits to aid in the quality of normal sleep patterns.

Although the use of sleeping pills and liquid sleep formulations have proven to be beneficial in alleviating sleep disorders, some sleeping pills and liquid sleep formulations can result in residual drowsiness, lethargy, hangover, tired, sluggish, and/or exhaustive feelings during wake hours. It is believed that these after-sleep or wake hours feelings are contributed to narcotic and/or hypnotic sleep agents that are contained in the sleeping pills or liquid sleep formulations. It has been shown that the incorporation of narcotic and/or hypnotic sleep agents can result in habituation, dependence, or addiction in addition to tolerance or withdrawal symptoms that can be associated with the sleeping pills or liquid sleep formulations containing such sleep agents.

For example, a known sleeping pill such as melatonin has been shown to provide for normal sleep patterns. Melatonin can be administered orally, intranasally, and/or topically to treat symptoms of sleep disorders. Melatonin, however, can be classified as a hypnotic sleep aid, thus the administration of melatonin can result in unwanted effects of feeling tired or sluggish during awakening hours.

Another example of sleeping aids that can provide for sleep benefits includes agents such as diphenhydramine and the benzodiazepenes. Diphenhydramine, however, is known as a sedative that has been shown to not only provide for effective treatment of symptoms related to sleep, but the administration of diphenhydramine can also result in a tired or sluggish feeling during awakening hours. The benzodiazepenes have been shown to provide dependency and addictive properties.

Therefore, the need exists for a method of promoting sleep by the administration of a sleeping aid that is non-hypnotic, and that provides for high quality sleep behaviors. It has been found that non-hypnotic vasoactive agents can be administered topically to provide quality sleep patterns with minimal perceived unwanted effects during wake hours.

The incorporation of non-hypnotics into pharmaceutical and cosmetic compositions is known. Exemplary non-hypnotics include compounds such as the vitamin B3 class of materials, a specific example of which includes the nicotinates. It has not, however, been demonstrated that non-hypnotics can be topically applied to provide for quality sleep benefits.

In fact, the topical application of sleep agents identified in prior disclosures includes the topical application of narcotic and hypnotic compounds. For example, U.S. Pat. No. 5,508,039 describes the transdermal administration of melatonin. WO 97/12612 discloses the administration of melatonin in combination with analgesic agents to relieve pain and induce sleep. U.S. Pat. No. 4,968,684 and EP 285,219 disclose a method of treating sleep disorders by using N-aryl-piperazinealkanamide derivatives.

It has been found that topical application of compositions comprising a non-hypnotic vasoactive agent is highly effective in promoting quality sleep. The topical administration of vasoactive agents, particularly non-hypnotic vasoactive agents, can result in the dilation of the blood vessels creating a phenomenon known as vasodilation. Accordingly, a method of promoting sleep by topically administering compositions comprising a non-hypnotic vasoactive agent that causes dilation of blood vessels (i.e., vasodilation) is beneficial in improving the quality of sleep.

### SUMMARY OF THE INVENTION

The present invention is directed to a method of promoting sleep by topically applying to mammalian skin a composition comprising a non-hypnotic vasoactive agent.

It has been found that the quality of sleep can be improved by topically applying compositions comprising a non-hypnotic, vasoactive agent, wherein the vasoactive agent provides for dilation of the blood vessels to result in normal sleep patterns. The method of application includes the topical application of the compositions to any area of the skin including, but not limited to, the head, face, arms, forearms, elbows, hands, stomach, buttocks, legs, knees, ankles, and feet.

### DETAILED DESCRIPTION OF THE INVENTION

The present invention is directed to a method of promoting sleep by the topical application of compositions comprising a non-hypnotic, vasoactive agent. The non-hypnotic, vasoactive agent provides for the dilation of blood vessels to result in improved quality of sleep.

The term “sleep disorders” as used herein refers to the onset, duration, and/or after effect of the lack of sleep. In other words, “sleep disorders” refer to, but is not limited to, the lack of ability to fall asleep, interrupted sleep, insomnia, lack of restfulness, on and off sleep, and the tired, sluggish, or exhaustive feeling due to the lack of sleep. The term “sleep” is typically referred to as the state of rest that occurs periodically and that is characterized by relative physical nervous inactivity, lessened responsiveness, unconsciousness, and typical brain wave patterns as measured using electroencephalography.

The term “hypnotic” as used herein refers to actives and drugs that induce sleep through direct central nervous system (CNS) depression, or other related CNS activity. Examples of hypnotics include, but are not limited to, the imidazopyridines, pyrazolopyrimidines, benzodiazepines, barbiturates, antianxiety drugs, opiods, sedating antihistamines, antipsychotics, tranquilizers, antiiolytics, and antidepressants. The term “non-hypnotic” as used herein refers to substances, compounds and materials that do not have direct CNS depressant effects, or other related CNS activity.

The term “vasoactive” as used herein refers to compounds and materials that can cause a dilation of blood vessels. It is understood that the term “vasoactive” includes constriction and dilation of blood vessels, however, as used herein the term “vasoactive” refers to the dilation of blood vessels.

The method of the present invention includes the topical application of compositions that can comprise, consist of, or consist essentially of the elements and limitations described herein, as well as any of the additional or optional ingredients, components, or limitations described herein.

All documents cited herein, including publications, patent applications, and issued patents mentioned herein, are, in relevant part, incorporated herein by reference. Citation of any document is not an admission regarding any determination as to its availability as prior art to the present invention.

### Vasoactive Agent

The present invention is directed to a method of promoting sleep, wherein the method involves the topical application of compositions that comprise a vasoactive agent. The vasoactive agent is non-hypnotic, and provides for the dilation of blood vessels to result in improved quality of sleep without any perceived negative after-sleep feelings of sedation, drowsiness, lethargy, hangover, tiredness and/or exhaustion. The non-hypnotic, vasoactive agent also provides for compositions that are not potentially habit forming, low tolerant, dependent, or can cause withdrawal symptoms.

The vasoactive agent can be included in the compositions as an individual vasoactive agent or as a combination of vasoactive agents, wherein the total concentration of the vasoactive agent typically ranges from about 0.001% to about 25%, preferably from about 0.01% to about 10%, more preferably from about 0.05% to about 5%, by weight of the composition.

Suitable vasoactive agents include vitamin B3 compounds, natural extracts, pyridine derivatives, circulation and cardiovascular compounds, and mixtures thereof. Vitamin B3 compounds are preferred.

Specific nonlimiting examples of vitamin B3 compounds which are suitable for use as a preferred vasoactive agent herein include the nicotinates. Specific nicotinates include, but are not limited to, benzyl nicotinate, inositol hexanicotinate, nicotinic acid, nicotinyl alcohol, xanthine nicotinate, methyl nicotinate, ethyl nicotinate, propyl nicotinate, isopropyl nicotinate, butyl nicotinate, isoamyl nicotinate, hexyl nicotinate, phenyl nicotinate, guaiacyl nicotinate, caffeine nicotinate, xanthinol nicotinate, nicametate citrate, pyridoxine, nicotinamide, nicotinuric acid, nicotinyl hydroxamate, tocopheryl nicotinate, panthenol, pantothenic acid, and mixtures thereof.

Specific nonlimiting examples of natural extracts suitable for use as a vasoactive agent herein include botanical extracts, herbal extracts, and mixtures thereof. Specific botanical and herbal extracts include, but are not limited to, Tang-kuei, Ho-shou-wu, Lycium fruit, Astrogalus, Psoralea, gelatin including the hide gelatin of Equus asinus, Polygonatum root, Codonopsis, Rehmannia, Eucommia, Dynaria, Cistanche, Cuscuta, Epimedium, asiatic acid, Genines amel, Centella asiatica, Rhizoma bark, Pitera, licorice extract, Lycium barbarum, gamma-oryzanol extracted from rice, and mixtures thereof.

Specific nonlimiting examples of pyridine derivatives suitable for use as a vasoactive agent herein include 1,10-phenanthroline; 2,2′-dipyridyl; 3,3′-dipyridyl; 2,3′-dipyridyl; 2,4′-dipyridyl; 4,4′-dipyridyl; 7-azaindole; octopirox; 1-(2-pyridyl)piperazine; 2,2′-dipyridylamine; 2-(2-aminoethyl)pyridine; phenanthridine; 2,2′-dipyridyl ketone; 1,2-bis(2-pyridyl)ethylene; 8-hydroxyquinoline N-oxide; 2,3-di-2-pyridyl-2,3-butanediol; 1,2-dianilinoethane; 2,3-bis(2-pyridyl)pyrazine; 2,3-bis(2-pyridyl)-5,6-dihydropyrazine; 3-hydroxypicolinamide; 2-(2-pyridyl)benzimidazole; 2-methyl-1,2-di-3-pyridyl-1-propanone; 1,4,8,12-tetraazacyclopentadecane; phenyl 2-pyridyl ketoxime; 3-(4-phenyl-2-pyridyl)-5-phenyl-1,2,4-triazine; 1-(3-aminopropyl)-imidazole; and mixtures thereof.

Specific nonlimiting examples of circulation and cardiovascular compounds suitable for use as a vasoactive agent herein include captopril, clemastine, arterenol, nicardipine, nifedipine, adenosine, khellin, lidocaine, nicotine, nitroglycerin, hydralazine, pipecolinic acid, quinidine, 3,5-pyridine dicarboxylic acid, dipyridamole, allopurinol, aminophylline, caffeine, caffeine benzoate, theophylline, and mixtures thereof.

### Compositions

The method of the present invention includes the topical application of the vasoactive agent described hereinabove. The vasoactive agent is typically incorporated into compositions that can be topically applied. The compositions include any known or otherwise effective composition that can be topically applied to mammalian skin. The terms “topical application” and “topically applied” are used interchangeably herein to refer to the application onto the outer layer of mammalian skin, which include, but is not limited to, application by rubbing onto the skin, brushing, painting, wiping, stroking, and the like.

Nonlimiting examples of compositions that can be topically applied include those known or otherwise effective compositions suitable for use in beauty, pharmaceutical, cosmetic, skin care technologies, and the like. Specific examples of such compositions include, but are not limited to moisturizers, lotions, astringents, facial and skin cleansers, body oils, ointments, gels, creams, and so forth. These compositions typically comprise a carrier system that contains the vasoactive agent.

The carrier system of the compositions defined herein include aqueous systems, non-aqueous systems, emulsion systems, and mixtures thereof. Specific examples of solvents that can be included in these carrier systems include, but are not limited to oils, alcohols, silicones, glycols, and mixtures thereof. Specific examples of suitable emulsion systems include, but are not limited to, oil-in-water emulsions, water-in-oil emulsions, oil-in-water-in-silicone emulsions, and mixtures thereof.

The compositions typically comprise the carrier system at concentrations ranging from about 75% to about 99.999%, preferably from about 90% to about 99.99%, more prefereably from about 95% to about 99.95%, by weight of the composition.

In addition to the carrier system, the compositions can comprise optional components that are known or otherwise effective for use in beauty, pharmaceutical, cosmetic, and skin care compositions, provided that the optional components are physically and chemically compatible with the vasoactive agent defined herein or do not otherwise unduly impair product stability, aesthetics, or performance. Optional components suitable for use herein include materials such as pH adjusting agents, preservatives, humectants, moisturizers, fragrances, surfactants, skin penetration enhancers, and so forth. The optional components are typically included at concentrations ranging from about 0.01% to about 50%, preferably from about 0.1% to about 10%, by weight of the composition.

### Method of Use

The present invention is directed to a method of promoting sleep by topically applying onto the skin of mammalians compositions that comprise a vasoactive agent defined herein. Typically a safe and effective amount of the composition is applied to the skin. In this context, the term “safe and effective amount” refers to an amount with provides a therapeutic benefit with minimal or no adverse reactions.

As referred to herein, the method of promoting sleep includes any known or otherwise effective method of promoting sleep patterns and behaviors that provide for the onset and/or duration of sleep, which includes the patterns and behaviors that are exhibited prior to falling asleep. Nonlimiting examples of such patterns and behaviors include the calm and/or relaxed feeling prior to falling asleep, uninterrupted sleep, restful sleep, drowsiness, slumber sleep, dozing, nodding, and mixtures thereof.

To promote sleep, compositions that comprise a vasoactive agent defined herein are topically applied onto mammalian skin. In general, from about 0.1 milligram of composition/square centimeter of skin (mg/cm²) to about 10 mg/cm² is topically applied to result in from about 0.1 mg to about 100 mg, preferably from about 1 mg to about 10 mg of vasoactive agent being applied onto the skin.

The compositions can be topically applied to any area of mammalian skin, including but not limited to, areas of the head, face, arms, forearms, elbows, hands, stomach, buttocks, legs, knees, ankles, feet, and mixtures thereof. The same or different amounts of the composition can be applied, provided that the total amount of composition being applied onto the skin does not exceed 10 mg/cm² for a single application process, wherein a single application process includes one or more applications onto one or more areas of mammalian skin.

The compositions can be applied using a single application or multiple application processes prior to falling asleep to improve the quality of sleep. The application processes can vary with the area of the skin onto which a composition is applied, for example an application process can involve the application of a composition onto the arm one or more times to result in improved quality of sleep, or alternatively one or more application processes can involve application onto the hands and/or legs one or more times to improve the quality of sleep. It is believed that the area of skin onto which a composition is applied will influence the amount of change in skin and/or core body temperature to result in a higher or lower propensity to fall asleep in addition to improved sleep patterns.

The compositions can be applied onto the skin using any known or otherwise effective application technique including, but not limited to, the techniques of rubbing, brushing, painting, wiping, and stroking a composition onto the skin. A highly effective method of promoting sleep has been developed by applying a composition onto the skin one or more times prior to bedtime, wherein the composition comprises a non-hypnotic, vasoactive agent.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Compositions that can be topically applied onto the skin to improve sleep quality are exemplified hereinbelow. The compositions comprise a non-hypnotic, vasoactive agent that is highly effective in providing for improved sleep benefits. The compositions can be applied onto one or more areas of the skin one or more times prior to bedtime to result in improved sleep quality.

### Example 1

Below is an example of a gel composition suitable for use in the method of the present invention. The gel composition is formed by combining and mixing the ingredients using known conventional gel forming processes. The gel is applied onto the outer skin surface of the hands and feet prior to bedtime.

| Ingredient | Weight % |
|---|---|
| Methyl nicotinate | 0.10 |
| SD alcohol 40 | 20.0 |
| Carbopol EDT 2001 | 1.00 |
| Sodium hydroxide, 50% | 0.60 |
| Deionized water | q.s to 100 |

### Example 2

Below is an example of a skin cream suitable for use in the method of the present invention. The skin cream is formed by combining and mixing the ingredients using known conventional processes for formulating skin creams. The skin cream is applied to skin areas of the lower legs and feet prior to bedtime.

| Ingredient | Weight % |
|---|---|
| Benzyl nicotinate | 0.20 |
| Carbopol 980 | 0.50 |
| Hydroxyethylcellulose | 0.50 |
| SD alcohol 40 | 20.0 |
| DC 556 silicone fluid | 2.00 |
| Witch hazel distillate | 0.20 |
| Sodium hydroxide, 50% | 1.00 |
| Fragrance | 0.10 |
| Deionized water | q.s. to 100 |

### Example 3

Below is an example of a skin cream suitable for use in the method of the present invention. The skin cream is formed by combining and mixing the ingredients using known conventional processes for formulating skin creams. The skin cream is applied to skin areas of the forearms and hands prior to bedtime.

| Ingredient | Weight % |
|---|---|
| Inositol hexanicotinate | 0.50 |
| Glycerine | 7.00 |
| Isododecane | 3.00 |
| Polyacrylamide, C13-C14 issoparaffin, Laureth-7 | 2.50 |
| Dimethicone, Dimethiconol | 2.00 |
| Isopropyl isostearate | 1.33 |
| Sorbitan monostearate, Sucrocoate | 1.00 |
| Cetyl alcohol | 0.72 |
| Tocopherol acetate | 0.50 |
| Panthenol | 0.50 |
| Stearyl alcohol | 0.50 |
| Deionized water | q.s. to 100 |

While particular embodiments suitable for use in the method of the present invention have been described, it will be obvious to those skilled in the art that various changes and modifications of the present invention can be made without departing from the spirit and scope of the invention. It is intended to cover, in the appended claims, all such modifications that are within the scope of this invention.

What is claimed is:

1. A method of promoting sleep by topically applying to mammalian skin a composition comprising a non-hypnotic vasoactive agent.

2. The method of claim 1 wherein the non-hypnotic vasoactive agent is selected from the group consisting of vitamin B3 compounds, natural extracts, pyridine derivatives, circulation and cardiovascular compounds, and mixtures thereof.

3. The method of claim 2 wherein the non-hypnotic vasoactive agent is a vitamin B3 compound.

4. The method of claim 3 wherein the vitamin B3 compound is selected from the group consisting of benzyl nicotinate, inositol hexanicotinate, nicotinic acid, nicotinyl alcohol, xanthine nicotinate, methyl nicotinate, ethyl nicotinate, propyl nicotinate, isopropyl nicotinate, butyl nicotinate, isoamyl nicotinate, hexyl nicotinate, phenyl nicotinate, guaiacyl nicotinate, caffeine nicotinate, xanthinol nicotinate, nicametate citrate, pyridoxine, nicotinamide, nicotinuric acid, nicotinyl hydroxamate, tocopheryl nicotinate, panthenol, pantothenic acid, and mixtures thereof.

5. The method of claim 1 wherein from about 0.1 mg/cm² to about 10 mg/cm² of the composition is topically applied to the mammalian skin.

6. The method of claim 5 wherein the composition is applied to areas of the mammalian skin selected from the group consisting of head, face, arms, forearms, elbows, hands, stomach, buttocks, legs, knees, ankles, feet, and mixtures thereof.
